(19) United States
(12) Patent Application Publication
(10) Pub. No.: US 2007/0202519 A1
(43) Pub. Date: Aug. 30, 2007

(54) Domain segmentation and analysis

(75) Inventors: Aibing Rao, Burlingame, CA (US); Eugeni A. Vaisberg, Foster City, CA (US)

Correspondence Address: BEYER WEAVER LLP, P.O. BOX 70250, OAKLAND, CA, 94612-0250, US

(73) Assignee: Cytokinetics, Inc.

(21) Appl. No.: 11/651,885

(22) Filed: Jan. 9, 2007

Related U.S. Application Data
(60) Provisional application No. 60/757,598, filed on Jan. 9, 2006.

(30) Foreign Application Priority Data
Mar. 8, 2006 (GB) 0604616.3

(51) Int. Cl. C12Q 1/68; G06F 19/00

(52) U.S. Cl. 435/6; 702/20

(57) ABSTRACT

Methods and apparatus, including computer program products, implementing and using techniques for analysis of images of cells and extraction of biologically significant features from the cell images, such as features located in cell boundary regions and cell-cell junctions. The extracted features may be correlated with particular conditions induced by biologically-active agents with which cells have been treated, thereby enabling the automated analysis of cells based on features that can be discovered in the cell boundary regions and cell-cell junctions of the images.

### CROSS-REFERENCE TO RELATED APPLICATIONS

This application claims priority under 35 U.S.C. § 119(e) to U.S. provisional application No. 60/757,598 filed on Jan. 9, 2006 and titled DOMAIN SEGMENTATION AND ANALYSIS, hereby incorporated by reference for all purposes. This application also claims priority under 35 U.S.C. § 119 to Great Britain application No. 0604616.3, filed Mar. 8, 2006 and also titled DOMAIN SEGMENTATION AND ANALYSIS, hereby incorporated by reference for all purposes. This application is related to US Patent Publication No. US 2005-0014217 A1 of Mattheakis et al., published Jan. 20, 2005, and titled “PREDICTING HEPATOTOXICITY USING CELL BASED ASSAYS,” and to US Patent Publication No. US 2005-0014216 of Mattheakis et al., published Jan. 20, 2005, and titled “PREDICTING HEPATOTOXICITY CELL BASED ASSAYS,” both of which are incorporated herein by reference for all purposes.

Provided are methods, apparatus and computer program products for analyzing images of biological systems such as individual cells.

Many interesting biological conditions can be correlated with features and conditions that occur on a cellular level. Biological “conditions” of interest to researchers include, for example, disease states, normal unperturbed states, quiescent states, states induced by exogenous biologically-active agents, and so on. Valuable insight may be gained by inducing a biological condition through a genetic manipulation, exposure to a particular agent (e.g., a compound, radiation, a field, and so on), deprivation of required substance, and other perturbations. Such a condition may cause changes in the occurrence and/or distribution of various proteins and other subcellular components within a cell. Conversely, detection of the presence and/or distribution of such proteins and subcellular components within the cell may be indicative of that particular condition.

In drug discovery, valuable information can be obtained by understanding how a potential therapeutic agent affects a cell. This information may give some indication of the mechanism of action associated with the compound. Understanding events that occur at the peripheral regions of a cell or at the junctions between two or more cells can provide valuable information about various cell conditions.

For example, hepatocytes are cells that make up 60-80% of the cytoplasmic mass of the liver. Hepatocytes are involved in protein synthesis, protein storage and transformation of carbohydrates, synthesis of cholesterol, bile salts and phospholipids, and detoxification, modification and excretion of exogenous and endogenous substances. Hepatocytes also initiate the formation and secretion of bile. At cell-cell junctions, hepatocytes form canalicular structures. Many ATP dependent transporters and other proteins are differentially localized in the canalicular membranes. Perturbations of the distribution and function of these proteins can be correlated with various types of drug-induced hepatotoxicity, as well as other types of conditions, such as cholestasis (a condition where bile is prevented from flowing from the liver to the duodenum) or phospholipidosis (an excessive accumulation of intracellular phospholipids).

Disclosed are methods and apparatus for the analysis of images of cells and extraction of biologically significant features from the cell images, for example, features located in cell boundary regions and cell-cell junctions. The extracted features may be correlated with particular conditions induced by biologically-active agents with which cells have been treated, thereby enabling the automated analysis of cells based on features that can be discovered in the cell boundary regions and cell-cell junctions of the images. In certain embodiments, methods for segmentation of cells in an image make use of data from separate images or channels of different cell components. Also disclosed are techniques for extraction of biologically relevant cell features from segmented cell images, for example, with respect to cell boundaries and cell-cell junctions.

In certain embodiments, image data for a reference cell component is used to segment cell peripheral regions (for example, cell nuclei and cell boundaries) is processed together with image data for a marker indicating a feature of interest located in a cell boundary region or a cell-cell junction (for example, cytoskeletal components (for example, acting), one or more markers indicating canalicular structures (for example, MRP2, a multidrug resistance protein 2 localized to canalicular membranes and transporting divalent bile salts and bulky organic conjugates, or BSEP, a bile salt export pump localized to canalicular membranes and exporting bile salt across the canalicular membranes)). Further, certain embodiments provide an analysis technique for extracting biologically relevant features from the cell boundaries and cell-cell junctions of the segmented images.

Certain embodiments provide methods and apparatus, including computer program products, implementing and using techniques for characterizing one or more cell features within one or more boundary regions of biological cells. An image of one or more cells is segmented to identify cell boundaries for the individual cells in the image. One or more boundary regions are defined for the individual cells in the image, based on the identified cell boundaries. One or more cell features within the one or more defined boundary regions are characterized.

Certain embodiments can include one or more of the following features. The image of one or more cells can be received, in which a nucleus-marker and a cell shape-indicative marker identify the nucleus and an overall cell shape for cells in the image. The image can include a digital representation of the one or more cells. The nucleus marker can be a DNA marker and the cell shape-indicative marker can be a non-specific protein marker or a cytoskeletal protein marker. The cells can be hepatocyte cells.

The image can further contain a marker for each of the one or more cell features within the one or more boundary regions. The marker for the one or more cell features can be selected from the group consisting of: an acting marker, an MRP2 marker, a BSEP marker, and a TGN marker. Segmenting can include segmenting the image using a watershed algorithm. Identifying boundary regions can include defining one or more of: a periphery region, a contact periphery region, a free periphery region, and a cell contact region, for the individual cells in the image. The periphery of a cell can be identified in the image as a subset of pixels inside the cell for which a mask with a predetermined size centered on each of the pixel covers at least one of the cell's boundary pixels. The contact periphery of a cell can be identified in the image as a subset of pixels inside the cell for which a mask with a predetermined size centered on each of the pixels covers at least one of the cell's boundary pixels and at least one boundary pixel of an adjacent cell. The free periphery of a cell can be identified in the image as a subset of pixels that are periphery pixels but not contact periphery pixels. The cell contact can be identified in the image as a pixels in a region between a pair of cells for which a mask with a predetermined size centered on each of the pixels covers at least one boundary pixel from each cell in the pair of cells. Characterizing one or more cell features can include characterizing an acting level in the boundary regions. It can be determined whether the cells possess and increased concentration of acting in the cell boundary regions.

Various biological conditions of interest involving changes or features of cell boundary regions or cell-cell junctions can be automatically identified through image processing techniques. Valuable insight of intra-cellular and inter-cellular mechanisms may be gained by inducing biological conditions through various mechanisms, such as genetic manipulation, exposure to a particular agent, deprivation of required substance, and other perturbations, and using the inventive image analysis to study the results. In certain embodiments the distributions of MRP2 and BSEP between a hepatocyte as a whole and its boundary regions can be used to indicate cholestasis. In certain embodiments, a redistribution of BODIPY® from the center of the cell to its periphery may indicate a steatotic effect.

The details of one or more embodiments are set forth in the accompanying drawings and the description below. Other features will be apparent from the description and drawings, and from the claims.

The patent or application file contains at least one drawings executed in color. Copies of this patent or patent application publication with color drawing(s) will be provided by the Office upon request and payment of the necessary fee.

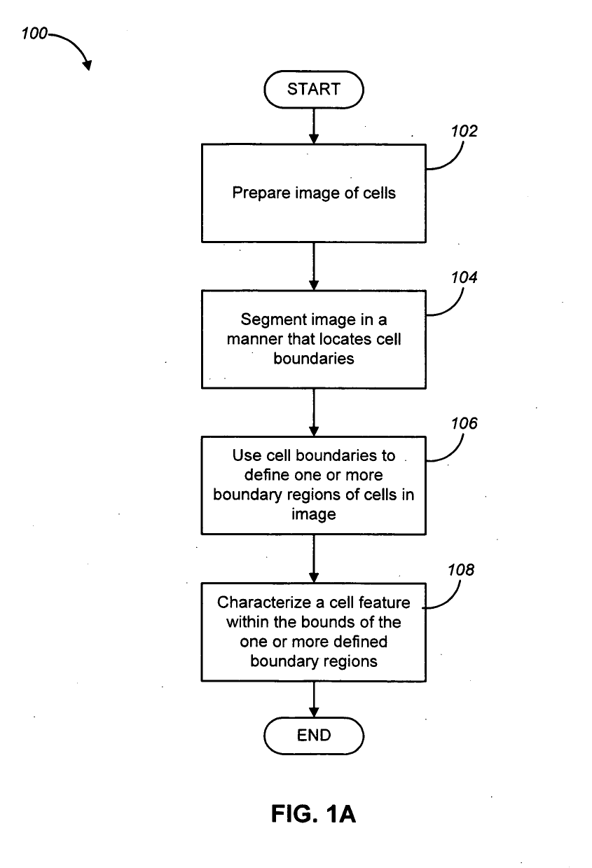

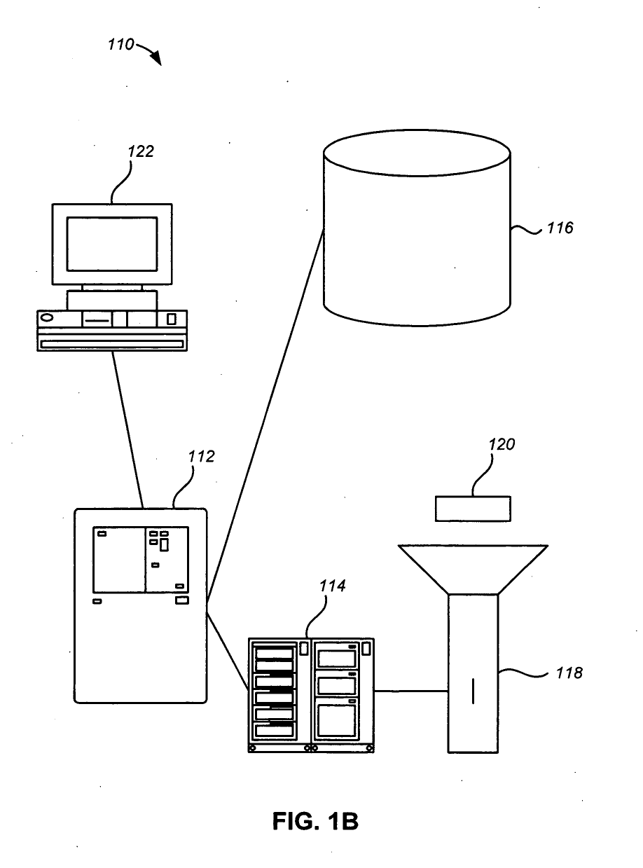

FIG. 1A is a flowchart showing an image analysis process in accordance with one embodiment.

FIG. 1B is a schematic block diagram of an image capture and image processing system.

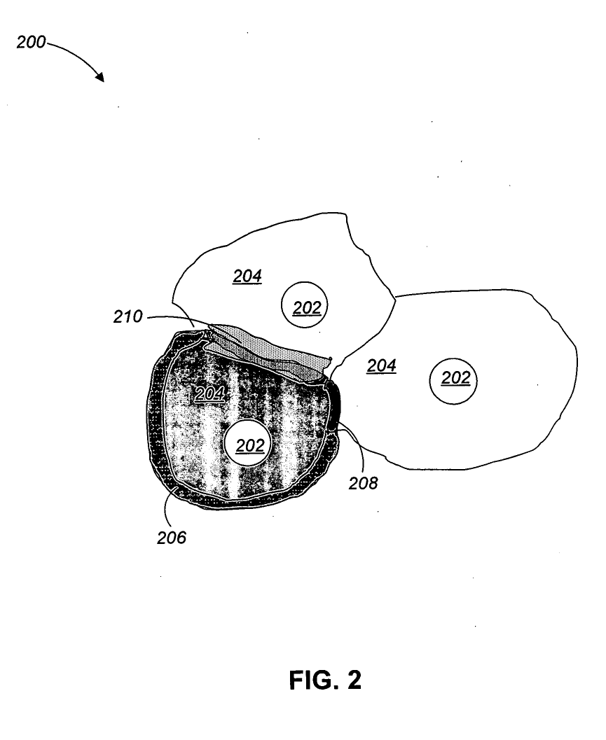

FIG. 2 is a schematic figure of a segmented cell image, showing three cells and various boundary regions identified within the segmented image.

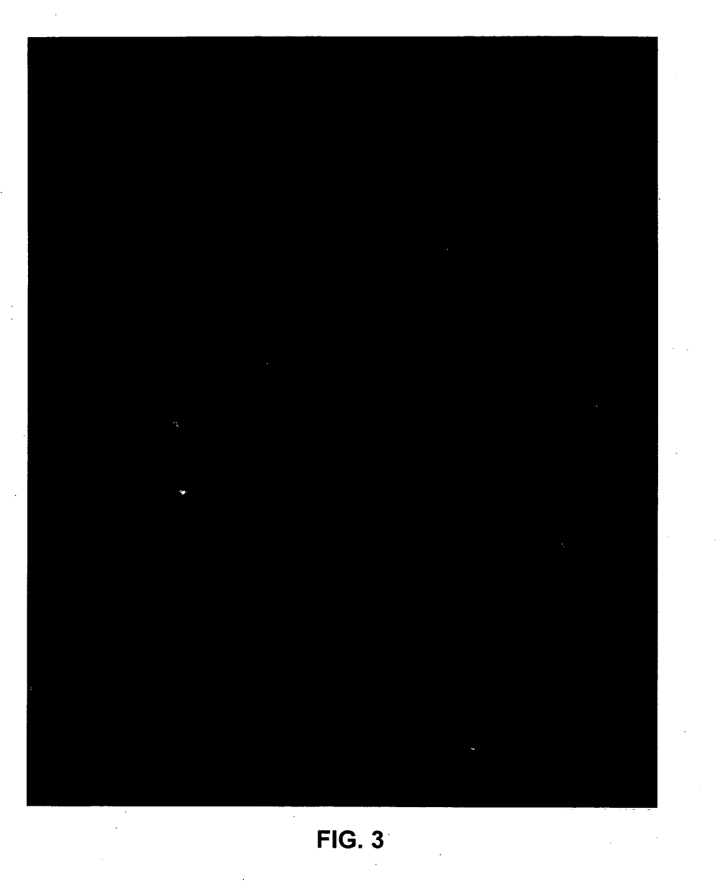

FIG. 3 is an exemplary image of hepatocyte components (red for DNA, blue for non-specific protein, and green for acting) taken from a sandwich culture.

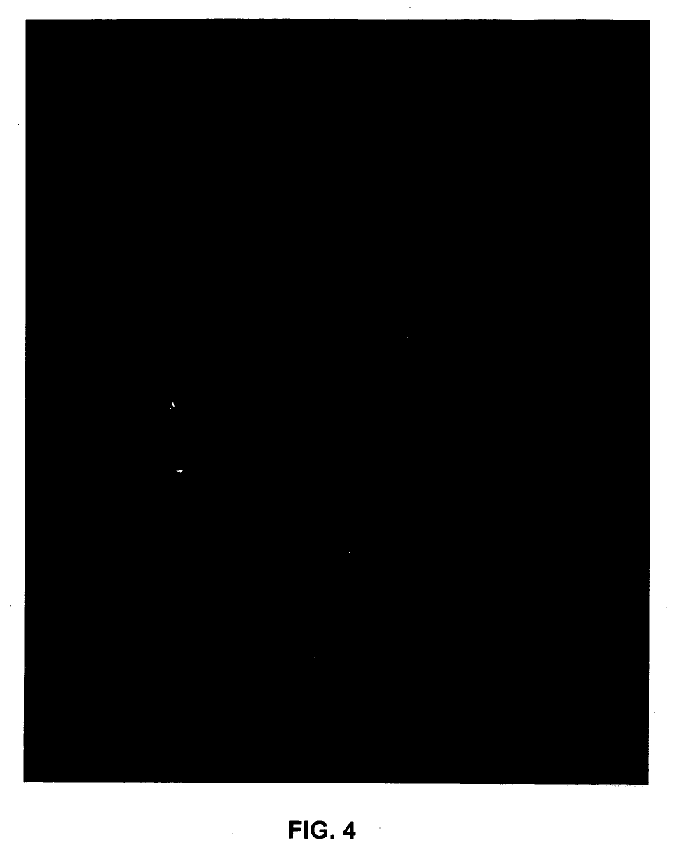

FIG. 4 is an examplary image of a nuclei mask for the hepatocyte sandwich culture in FIG. 3.

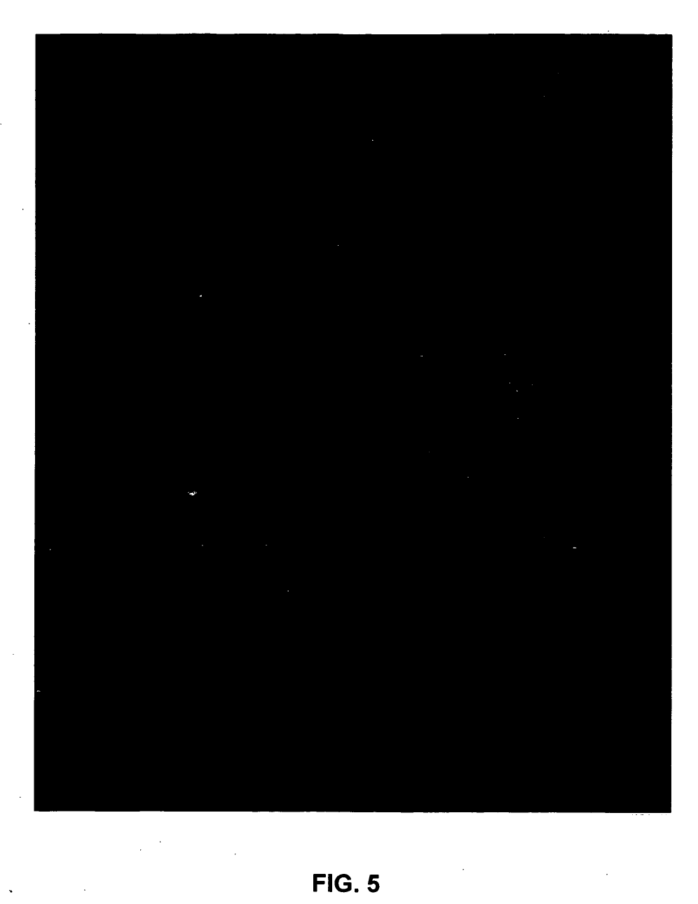

FIG. 5 is an exemplary image of a cell mask for the hepatocyte sandwich culture in FIG. 3.

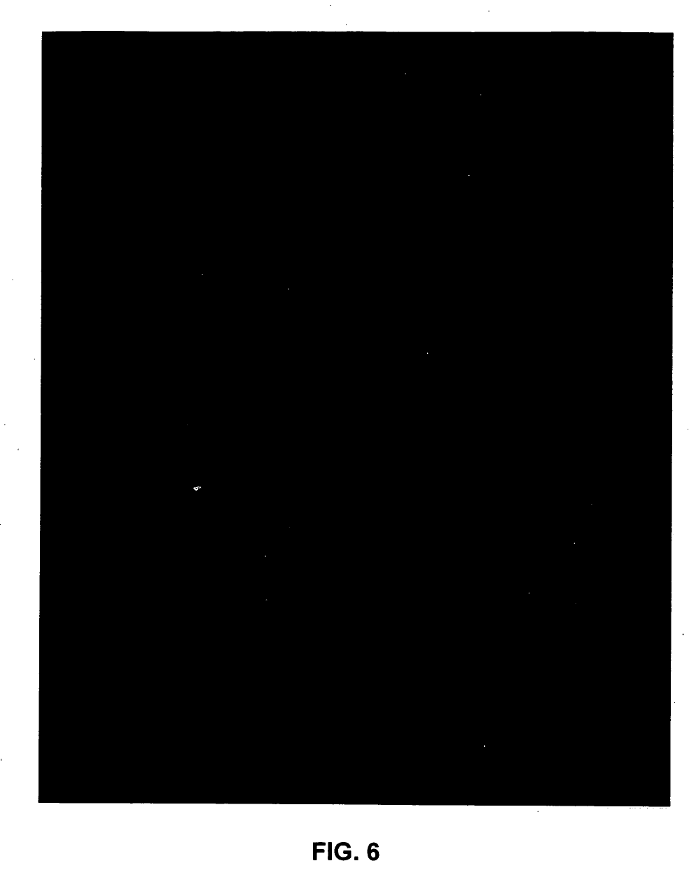

FIG. 6 shows the image of the hepatocytes in FIG. 3, with a periphery mask applied.

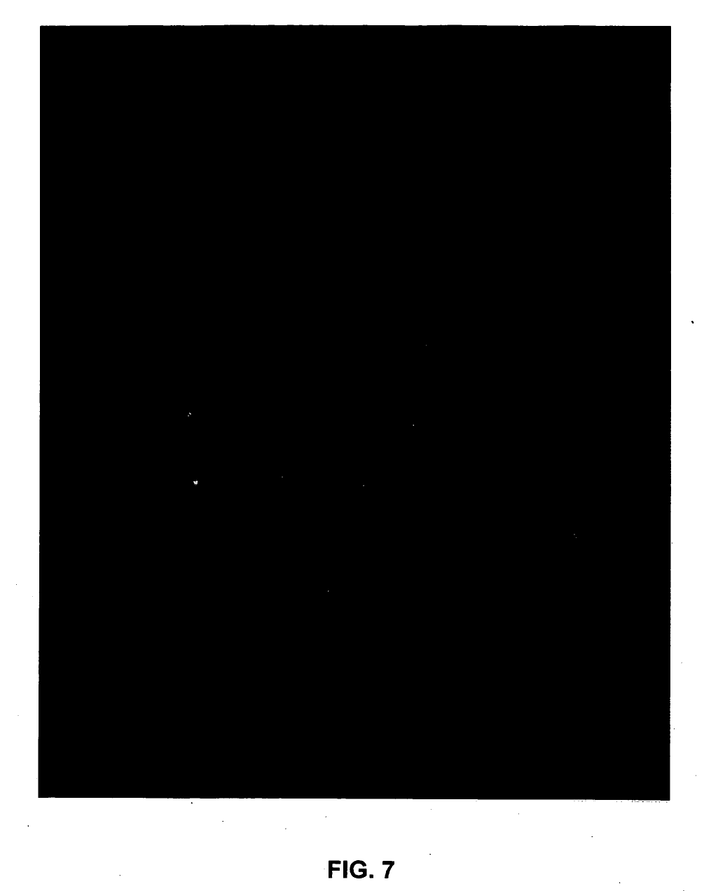

FIG. 7 shows the image of the hepatocytes in FIG. 3, with a free periphery mask applied.

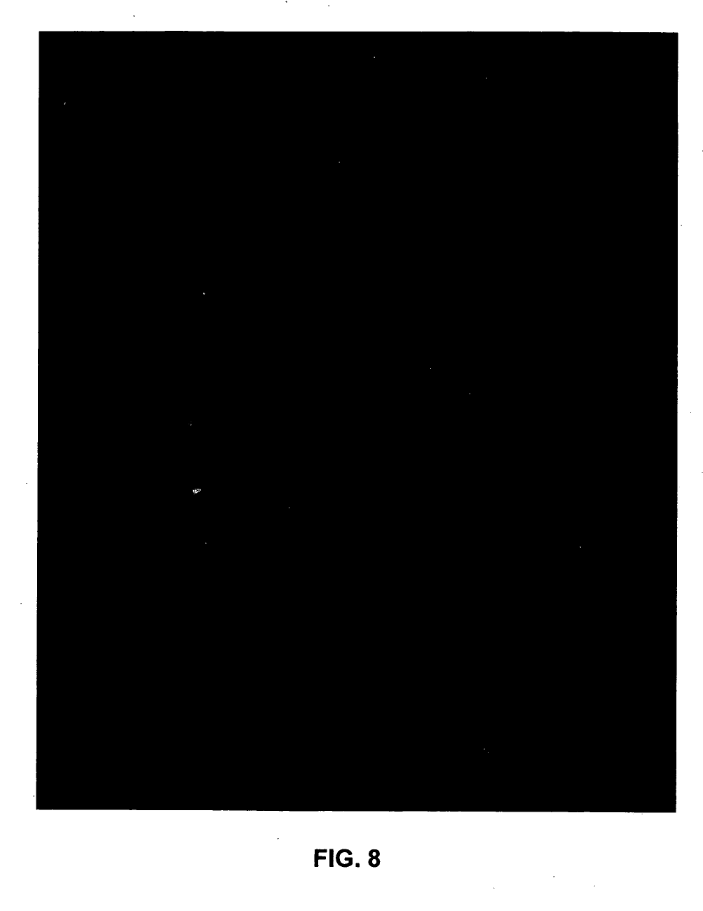

FIG. 8 shows the image of the hepatocytes in FIG. 3, with a contact periphery mask applied.

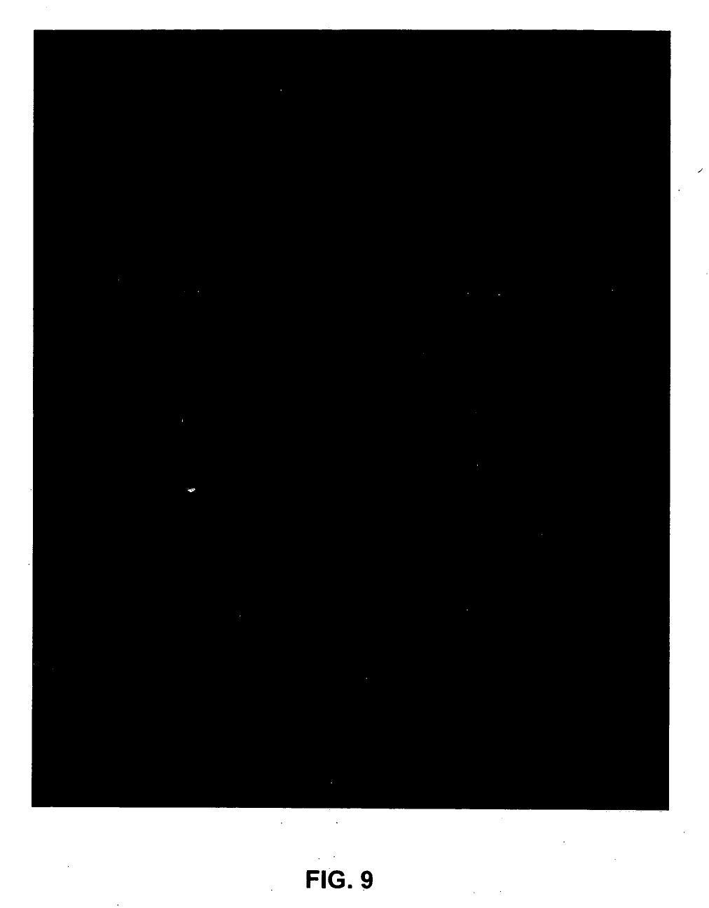

FIG. 9 shows the image of the hepatocytes in FIG. 3, with a cell contact mask applied.

Like reference symbols in the various drawings indicate like elements.

Generally, this disclosure relates to image analysis processes and apparatus configured for image analysis. It also relates to machine-readable media on which is provided instructions, data structures, and so on, for performing the processes described herein. In accordance with certain embodiments, images of cells are manipulated and analyzed in certain ways to extract relevant intra-cellular and inter-cellular features. Using those features, certain conclusions about the biology of a cell or a group of cells can be automatically drawn.

Provided are methods and apparatus for analysis of images of cells and extraction of biologically significant features from the cell images, such as cell boundary regions and cell-cell junctions. The extracted features may be correlated with particular conditions induced by biologically-active agents with which cells have been treated, thereby enabling the automated analysis of cells based on features that can be viewed in the cell boundaries and cell-cell junctions in the images. Provided are methods for segmentation of cells in an image using data from several separate images of different cell components. Further provided are techniques for extraction of biologically relevant cell features from segmented cell images, for example, with respect to cell boundaries and cell-cell junctions.

In certain embodiments, image data for a reference cell component is processed to identify boundaries of individual cells. A watershed algorithm may be employed for this purpose. See U.S. Pat. No. 6,956,961 of Cong, et al., titled “EXTRACTING SHAPE INFORMATION CONTAINED IN CELL IMAGES,” which is incorporated herein by reference for all purposes. The cell boundaries may then be processed to identify one or more peripheral regions (as described elsewhere herein). These boundaries may then be used together with image data for a marker relevant a condition at a free cell boundary region or a cell-cell junction (for example, cytoskeletal components (for example, acting), one or more markers indicating canalicular structures (for example, MRP2, a multidrug resistance protein 2 localized to canalicular membranes and transporting divalent bile salts and bulky organic conjugates, or BSEP, a bile salt export pump localized to canalicular membranes and exporting bile salt across the canalicular membranes)). As indicated, this disclosure provides analysis techniques for extracting biologically relevant features from the cell boundary regions and cell-cell junctions of the segmented images.

Examples of specific embodiments are illustrated in the accompanying drawings. While the apparatus and methods are described in conjunction with these specific embodiments, it will be understood that this description is not intended to be limited to the described embodiments. On the contrary, the description is intended to cover alternatives, modifications, and equivalents as may be included within the spirit and scope of the appended claims. In the following description, specific details are set forth in order to provide a thorough understanding of the apparatus and methods. The apparatus and methods can be practiced without some or all of these specific details. In addition, well-known features or details may not have been described to avoid unnecessarily obscuring the essential features of the apparatus and methods.

FIG. 1A shows a flowchart of a process (100) for obtaining and processing images. As can be seen in FIG. 1, the process starts with preparation of an image of cells to be analyzed (step 102). The image is then segmented so that the cell boundaries can be identified within the image (step 104). Once the cell boundaries have been defined, one or more “boundary regions” of the cells in the image are defined (step 106). These boundary regions may be identified by various techniques (as described below) depending upon the particular category or type of boundary region to be identified. Examples include free boundary regions, where a cell does not contact any other cell, or contact boundary regions, where two cells contact each other.

In certain embodiments, segmentation in step 104 and the identification of boundary regions in step 106 may be accomplished by traversing the entire image one or more times, pixel-by-pixel, to define cell boundaries (segmentation step 104), and then traversing the entire image a second time to define boundary regions based on proximity to one or more cell boundaries (boundary region identification step 106). In certain embodiments, the boundary regions can be identified on a cell-by-cell basis. After one or more categories of boundary region are identified as appropriate, one or more cell features are characterized within the bounds of these one or more boundary regions (step 108). The identified features may then be used to further characterize and draw conclusions about various biological conditions. Each of the steps of the process (100) in FIG. 1A will now be discussed in further detail, followed by some illustrative examples that show various features that can be detected in accordance with certain embodiments.

Images may be obtained of cells that have been treated with a chemical agent to render visible (or otherwise detectable in a region of the electromagnetic spectrum) a cellular component. A common example of such agents are colored dyes specific for a particular cellular component that is indicative of cell periphery features, which enables the identification of cell boundary regions and cell-cell junctions that can be further analyzed. Other such agents may include fluorescent, phosphorescent or radioactive compounds that bind directly or indirectly (e.g., via antibodies or other intermediate binding agents) to a cell component. In certain embodiments, several cell components may be treated with different agents and imaged separately. Some of these may be useful for identifying cells and cell boundaries (e.g., DNA markers to identify nuclei and non-specific cell protein markers to identify cell boundaries).

Generally the images used as the starting point for the techniques disclosed herein are obtained from cells that have been specially treated and/or imaged under conditions that contrast markers of cellular components of interest from other cellular components and the background of the image. In a certain embodiments, the cells are fixed and then treated with a material that binds to a marker for the components of interest and shows up in an image. The chosen imaging agent can be chosen to bind indiscriminately with the marker, regardless of its location in the cell. The agent should provide a strong contrast to other features in a given image. To this end, the agent should be luminescent, radioactive, fluorescent, etc. Various stains and fluorescent compounds may serve this purpose.

A variety of imaging agents are available depending on the particular marker, and agents appropriate for labeling cytoskeletal, cytoplasmic, plasma membrane, nuclear, and other discrete cell components are well known in the histology art. Examples of such compounds include fluorescently labeled antibodies to cytoplasmic or cytoskeletal proteins, fluorescent dyes which bind to proteins and/or lipids, labeled ligands which bind to cell surface receptors, and fluorescent DNA intercalators and fluorescently labeled antibodies to DNA or other nuclear component which bind to the nuclei. For example, a suitable label for the cytoskeletal protein tubulin is a fluorescently labeled monoclonal antibody to tubulin, rhodamine-labeled DM1alpha, produced from hybdridoma DMlA reported in the publication Blose et. al. Journal of Cell Biology, V98, 1984, 847-858. Examples of fluorescent DNA intercalators include DAPI and Hoechst 33341 available from Invitrogen Inc. of Carlsbad, California. The antibodies may be fluorescently labeled either directly or indirectly. Other useful markers may be employed to image overall protein content within cell. One example is the Alexa 647 succinimidyl ester (Alexa 647) available from Invitrogen Inc. of Carlsbad, Calif. (a non-specific marker for free amine groups in proteins).

Cells may be treated with more than one imaging agent, each imaging agent specific for a different cellular component of interest. The component(s) may then be separately imaged by separately illuminating the cells with an excitation frequency (channel) for the imaging agent of the marker for the component of interest. Thus different images of the same cells focusing on different cellular components may be obtained on different channels, and imaged in the same resulting image, if so desired.

Various techniques for preparing and imaging appropriately treated cells are described in U.S. Pat. application Ser. No. 09/310,879 by Vaisberg et al., filed May 14, 1999 and titled “DATABASE METHOD FOR PREDICTIVE CELLULAR BIOINFORMATICS,” which is incorporated herein by reference in its entirety and for all purposes, and in U.S. Patent Application Publication No. US 2005-0014217 A1, which was incorporated above.

The assays described herein can be carried out in many different apparatuses. Generally, the cell samples are provided as discrete cell cultures on one or more support structures. Depending on the type of support structure, the cells may grow in two-dimensions or three-dimensions. Examples of support structures include bare plastic supports that include nutrients, glass surfaces, extra-cellular matrices such as collagen or Matrigel (available from BD Biosciences, San Jose, Calif.), etc. Such structures can be provided in multiwell plates, such as 24-, 96-, or 384-well assay plates (e.g., Costar plates (Corning Life Sciences, New York, N.Y.) among others). An assay plate is a collection of wells arranged in an array with each well holding multiple cells which are exposed to a stimulus or which provide a control sample. In other embodiments, single sample holders can be used instead of multi-well plates.

Preparation of cell cultures is well known and will not be described in detail here. Available standard cell cultures (e.g., HUVEC, A549, A498, DU145, SKOV3 and SF268) may be suitable for some applications. However, in other applications such as those involving studies of a tumor from a particular patient, the cells may be obtained from a biopsy. Procedures for extracting, plating and culturing such cells are well known. In certain embodiments, hepatocytes are employed and these may be prepared and imaged as described in US Patent Publication No. US 2005-0014217 A1, previously incorporated by reference.

FIG. 1B shows a schematic block diagram of an image capture and image processing system (110) which can be used to capture and process the images of cells and store cell counts, phenotypic data, and other information used in boundary domain analyses described herein. This diagram is merely a non-limiting example. The depicted system (110) includes a computing device (112), which is coupled to an image processor (114) and is coupled to a database (116). The image processor receives information from an image-capturing device (118), which includes an optical device for magnifying images of cells, such as a microscope. The image processor and image-capturing device can collectively be referred to as the imaging system herein. The image-capturing device obtains information from a plate (120), which includes several wells providing sites for groups of cells. The computing device (112) retrieves the information, which has been digitized, from the image-processing device and stores such information into the database (116).

A user interface device (122), which can be a personal computer, a workstation, a network computer, a personal digital assistant, or the like, is coupled to the computing device. In the case of cells treated with a fluorescent marker, a collection of such cells is illuminated with light at an excitation frequency from a suitable light source such as a halogen-lamp, arc lamp or laser (not shown). A detector part of the image-capturing device is tuned to collect light at an emission frequency. For example, this can be a digital camera that is sensitive to light over a wide range of frequencies. One may use emission filters to control which light wavelengths hits the camera. Examples of suitable cameras are the Orca-100 from Hamamatsu (Hamamatsu City, Japan) or the CoolSNAPH_{HQ}™ from Roper Scientific. The collected light is used to generate an image that highlights regions of high marker concentration.

The depicted apparatus also includes a fluidics system for providing fluid to individual cell samples on the support. Such system can be employed to deliver a compound or other treatment to individual cell samples. An example is the fluidics system on the live cell imaging addition of the Axon Imagexpress (Axon Instruments/Molecular Devices Corporation, Union City, Calif.).

In one embodiment individual pipettes are provided for the individual wells of a support. Metered doses of a compound under investigation or a washing fluid are provided to each of the individual wells or to groups of individual wells. The fluidics control system allows precise control of the drug wash off timing and flow conditions. The fluidics control system allows fine control of fluid flow rates, delivery times, aspiration rates, and separation distance of the pipette or other delivery nozzle from the wells.

The apparatus may also allow careful control of illumination conditions. Obviously when fluorescent markers are used the apparatus must be able to illuminate at appropriate excitation frequencies and capture radiation at the signature emission frequencies. However, it may also be important to ensure that the illumination conditions do not kill cells. Phototoxicity is a consideration. Imaging parameters to be optimized include the intensity of illumination (which may dictate magnification) and the frequency at which individual images are captured. Because different types of cells and different treatment regimens lead to different levels of sensitivity, systems allowing flexible illumination conditions may be used.

Other apparatus features include, optionally, mechanisms for controlling the environment in which the cells grow. Thus, the apparatus may include sub-systems for monitoring and controlling temperature and the atmospheric composition (e.g., carbon dioxide levels).

In the example of hepatocytes, as discussed above, the following markers may be of interest, as they indicate canalicular structures. One marker is TGN, which is an antibody marker for a protein P38, which accumulates in the Golgi apparatus of the cells. Whereas the Golgi apparatus typically is located relatively close to the nucleus in most cells, it is located close to the area of contact between cells in the case of hepatocytes. Being able to visualize the Golgi apparatus in hepatocytes can provide useful information about the status of the hepatocytes. Another marker is MRP2, which is antibody that binds to a multidrug resistance protein 2 localized to canalicular membranes. Yet another marker is BSEP, which is an antibody that binds to the bile salt export pump localized in the canalicular membranes. The cytoskeletal protein acting is another cellular component implicated in hepatocyte pathologies manifested by certain cell boundary phenotypes, so various acting markers may also be very useful in certain boundary domain assays.

An exemplary image of a hepatocyte sandwich culture can be seen in FIG. 3. The image in FIG. 3 shows an overlay of three channels (red, green and blue), which each indicates a particular feature of the cells. The red channel shows DNA, that is, primarily the nuclei of the cells, which have been stained using the Hoechst 33341 marker (available from Invitrogen Inc. of Carlsbad, Calif.) mentioned previously. The blue channel shows an Alexa 647 marker, which is a general protein marker for identifying cells (also identified above). The green channel shows the presence of Acting in the hepatocyte culture. As can be seen in FIG. 3, the acting appears to be primarily located in the regions between the cells, as indicated by virtue of its position with respect to the red nuclei and the long slender structures it produces. Other markers that may be of interest for boundary domain assays include Cytochrome C, BODIPY®, and Lysotracker, also available from Invitrogen Inc.

In order to derive biologically meaningful information from an analysis of cell images, it is important to be able to distinguish the individual cells from each other by establishing the cell boundaries. This process is often referred to as “segmentation.” Segmentation can be performed by various techniques including those that rely on identification of discrete nuclei and those that rely on the location of cytoplasmic proteins or cell membrane proteins. Exemplary segmentation methods are described in US Patent Publication No. US-2002-0141631-A1 of Vaisberg et al., published Oct. 3, 2002, and titled “IMAGE ANALYSIS OF THE GOLGI COMPLEX,” and US Patent Publication No. US-2002-0154798-A1 of Cong et al. published Oct. 24, 2002 and titled “EXTRACTING SHAPE INFORMATION CONTAINED IN CELL IMAGES,” both of which are incorporated herein by reference for all purposes.

Segmentation may be performed by first segmenting the DNA image, that is, the red channel in FIG. 3. This segmentation is done in order to convert the image into discrete regions/representations for the DNA of each nucleus to generate a “nuclei mask.” In certain embodiments, each representation includes only those pixels where the DNA of a single cell nucleus is deemed to be present. Any suitable stain for DNA or histones may work for this purpose (e.g., the DAPI and Hoechst stains mentioned above). Since the DNA is normally contained almost entirely within the nucleus of eukaryotic cells, the shape of each representation resulting from segmentation represents the boundaries within which a nucleus lies. The nuclei mask is a composite of the discrete nucleus representations providing intensity as a function of position for each nucleus in the image.

Individual cell nuclei may be identified in the image by various image analysis procedures. Exemplary approaches include edge finding routines and thresholding routines. Some edge finding algorithms identify pixels at locations where intensity is varying rapidly. For many applications of interest here, pixels contained within the edges will have a higher intensity than pixels outside the edges. Thresholding algorithms convert all pixels below a particular intensity value to zero intensity in an image subregion (or the entire image, depending upon the specific algorithm). The threshold value is chosen to discriminate between nucleus (DNA) images and background. All pixels with intensity values above threshold in a given neighborhood are deemed to belong to a particular cell nucleus. A detailed description of how to generate the nuclei mask can be found in U.S. Pat. No. 6,956,961, previously incorporated by reference.

FIG. 4 shows an example of a nuclei mask which was obtained in accordance with the techniques described above. The nuclei mask has been overlaid on the composite hepatocyte image in FIG. 3, and the identified nuclei are visible as little red circles in FIG. 4. The second part of this segmentation is to identify the remainder (that is, the non-nucleus part) of each individual cell, which will now be described.

In one embodiment, this cell mask is obtained by segmenting the image in the blue channel of FIG. 3, using a watershed algorithm. The concepts underlying watershed algorithms are well known. In some embodiments, a non-specific protein marker such as Alexa 647 stain (shown in the blue channel) provides the required “container component” and the cell nuclei identified above provide the required “seeds.” In another embodiment, a marker for a cell membrane protein is used with the cell nuclei to identify cell boundaries. When segmenting images of hepatocytes, it may be desirable to employ DM1α as the marker for an extra-nuclear component (tubulin). Given these parameters, one of skill in the art can apply known algorithms such as watershed algorithms to the cell image data in order to elucidate cell boundaries and thereby achieve segmentation of the cells in the image. Appropriate watershed algorithms suitable for use are described in detail in L. Vincent and P. Soille, Watersheds in digital spaces: an efficient algorithm based on immersion simulations, IEEE Transactions on Patter Analysis and Machine Intelligence, 13:583-589, 1991, incorporated by reference herein for all purposes. Further details about the application of the watershed algorithms are also described in U.S. Pat. No. 6,956,961, which was incorporated by reference above. FIG. 5 shows an image of the hepatocytes in FIG. 3, with the cell masks superimposed, as obtained by the above described watershed algorithm. It should be noted that the boundaries of the cells, and hence the cells' shapes are clearly delineated. Now that the cell boundaries have been identified, they will be further classified, as will be described below, in order to distinguish boundary regions that represent “free” boundaries and boundary regions that represent parts of boundaries that have neighboring cells.

In order to define “interesting” regions, from which signals will be analyzed, the cell boundaries in the segmented cell image of FIG. 5 are divided into a number of boundary regions. FIG. 2 shows a schematic view of a group of cells (three cells) (200) and their associated boundary regions. As can be seen in FIG. 2, each cell has a nucleus (202) and a region around the nucleus that is identified as non-periphery cytoplasm (204). For the boundary of a cell, the periphery, contact periphery, free periphery, and cell contact boundary regions are identified. In one embodiment, the boundary of a segmented object such as a nucleus or a cell object is defined as follows: a pixel in the object is defined as a boundary pixel if and only if any pixel among its surrounding eight pixels is not in the object.

The “periphery” of a cell is defined as a circular region inside a cell and having a defined width, e.g., 3 pixels depending upon magnification and image resolution (pixel density). In some embodiments, a pixel is labeled as a periphery pixel if a 3×3 pixel mask centered on the pixel covers a boundary pixel of the cell. Equivalently, based on the above definition of cell boundary, a pixel in the cell is a periphery pixel if a 3×3 pixel mask centered on the pixel covers at least one pixel outside of the cell. The periphery pixel is labeled as being a periphery pixel for the cell with the nearest cell boundary. In some embodiments, an entire segmented image is traversed with a 3×3 pixel mask. Whenever, the mask touches a boundary pixel (as identified by segmentation), the pixel on which the mask is centered is deemed part of the cell periphery. FIG. 6 shows the image of the hepatocytes in FIG. 3, with a periphery mask applied. As can be seen in FIG. 6, a three pixel wide “ring” around each cell can be distinguished, compared to the cell mask in FIG. 5, which merely is a single pixel curve showing the outline of the cells. It should be noted that the 3-pixel wide periphery mask is applied here to an image that was taken at 10× magnification and that has a resolution of one micron per pixel in both the horizontal and vertical directions. The width of the periphery mask will, of course, vary depending on the magnification of the microscope, the marker used, and based on other factors that affect the size of the cells in the image.

Returning to FIG. 2, the “contact periphery” (208) is defined as a subset of the periphery of which a 3×3 mask covers at least the boundary of two cells. In more detail, recalling the above definition of periphery, it requires that a 3×3 pixel mask centered on a periphery pixel cover at least one pixel outside of the cell. As the skilled person realizes, there are two cases; either the pixels of the mask that are outside of the cell are all background pixels, or some of the pixels of the mask that are outside of the cell are pixels that belong to other cells. In the second case, the periphery pixel is referred to as a contact periphery pixel. The first case corresponds to “free periphery” which will be discussed in the following paragraph. FIG. 8 shows the image of the hepatocytes in FIG. 3, with a contact periphery mask applied. As can be seen in FIG. 8, many of the “rings” around the cells in FIG. 6 have been broken up into segments, whereas other “rings” remain the same as in FIG. 6. The contact periphery of a cell can be viewed as those portions of the cell periphery associated with a boundary of the cell that contacts other cells. Again, it should be noted that the 3-pixel wide contact periphery mask is applied here to an image that was taken at 10× magnification and that has a resolution of 1 micron per pixel in both the horizontal and vertical directions. The width of the contact periphery mask will, of course, vary depending on the magnification of the microscope, the marker used, and based on other factors that affect the size of the cells in the image.

Returning again to FIG. 2, the “free periphery” (206) is defined as the subtraction of the contact periphery (208) from periphery region; that is, the part of the periphery where the cells do not contact any other cells. FIG. 7 shows the image of the hepatocytes in FIG. 3, with a free periphery mask applied. As can be seen in FIG. 7, the segments of the “rings” that were eliminated in FIG. 8, are displayed. The sum of the contact periphery regions and the free periphery regions in an image give the full periphery regions of the cells in the image. As before, it should be noted that the 3-pixel wide free periphery mask is applied here to an image that was taken at 10× magnification and that has a resolution of 1 micron per pixel in both horizontal and vertical direction. The width of the free periphery mask will, of course, vary depending on the magnification of the microscope, the marker used, and based on other factors that affect the size of the cells in the image.

Returning yet again to FIG. 2, the “cell contact” (210) is defined as the region between a pair of cells, in which a pixel is labeled as a cell contact pixel if a 3×3 mask (magnification and image resolution dependent) centered on the pixel covers both a boundary pixel of one cell and a boundary pixel of at least one other cell. The contact region may extend beyond the cell periphery into spaces between pairs of cells in certain cases. FIG. 9 shows the image of the hepatocytes in FIG. 3, with a cell contact mask applied. As can be seen in FIG. 9, these cell contact regions (210) contain significant amounts of acting (colored green in FIG. 9), which may be a biologically relevant result.

After one or more boundary regions have been identified, the presence of various markers within these individual regions can be used to characterize various cell features or conditions within these boundary regions. Similar to nuclei and cell masks, two categories of features can be defined for the above masks: intensity features and morphological features. Intensity features include, for example, total, mean, or maximum intensity of a marker within a boundary region, and the distribution of that marker within the boundary region (standard deviation, skewness, kurtosis, and so on.). Morphological features include the shape of the boundary region, such as area, perimeter, extent, solidity, axis ratio, eccentricity, major and minor axes, orientation, and the presence and/or condition of granules or other substructures within the boundary regions, and the like.

In some situations, as can be seen in FIG. 9, the cell contact regions (210) between the hepatocytes contain significant concentrations of acting, which is an indicator of the presence of canalicular structures. Thus, an increase of acting in the cell boundary regions or in the cell-cell junctions can be an indicator of enhanced inter-cellular communication, which in turn may be correlated with various biological conditions. Conversely, a decrease of acting in the cell boundary regions or in the cell-cell junctions can be an indicator of decreased inter-cellular communication, which may be correlated with other types of biological conditions.

Intensity changes of the markers within particular boundary regions may indicate certain pathological conditions in hepatocytes. For example, when the quotient of the mean intensities for cell contact BSEP divided by cell contact MRP2 changes, while the quotient of the mean intensities for the entire cell BSEP divided by the entire cell MRP2 remains constant, this is an indicator of cholestasis.

The apparatus and methods described herein can be implemented in digital electronic circuitry, or in computer hardware, firmware, software, or in combinations of them. The apparatus can be implemented in a computer program product tangibly embodied in a machine-readable storage device for execution by a programmable processor; and method steps can be performed by a programmable processor executing a program of instructions to perform cellular boundary region identification and processing algorithms by operating on input data (e.g., images in a stack) and generating output (e.g., mechanisms of action for certain compounds). One or more computer programs can be provided that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Suitable processors include, by way of example, both general and special purpose microprocessors. Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs (application-specific integrated circuits).

To provide for interaction with a user, a computer system having a display device such as a monitor or LCD screen for displaying information to the user can be provided. The user can provide input to the computer system through various input devices such as a keyboard and a pointing device, such as a mouse, a trackball, a microphone, a touch-sensitive display, a transducer card reader, a magnetic or paper tape reader, a tablet, a stylus, a voice or handwriting recognizer, or any other well-known input device such as, of course, other computers. The computer system can be programmed to provide a graphical user interface through which computer programs interact with users.

Finally, the processor optionally can be coupled to a computer or telecommunications network, for example, an Internet network, or an intranet network, using a network connection, through which the processor can receive information from the network, or might output information to the network in the course of performing the above-described method steps. Such information, which is often represented as a sequence of instructions to be executed using the processor, may be received from and outputted to the network, for example, in the form of a computer data signal embodied in a carrier wave. The above-described devices and materials will be familiar to those of skill in the computer hardware and software arts.

It should be noted that various computer-implemented operations involving data stored in computer systems are employed. These operations include, but are not limited to, those requiring physical manipulation of physical quantities. Usually, though not necessarily, these quantities take the form of electrical or magnetic signals capable of being stored, transferred, combined, compared, and otherwise manipulated. The operations described herein can be useful machine operations. The manipulations performed are often referred to in terms, such as, producing, identifying, running, determining, comparing, executing, downloading, or detecting. It is sometimes convenient, principally for reasons of common usage, to refer to these electrical or magnetic signals as bits, values, elements, variables, characters, data, or the like. It should remembered however, that all of these and similar terms are to be associated with the appropriate physical quantities and are merely convenient labels applied to these quantities.

Also provided are a device, system or apparatus for performing the aforementioned operations. The system may be specially constructed for the required purposes, or it may be a general-purpose computer selectively activated or configured by a computer program stored in the computer. The processes presented above are not inherently related to any particular computer or other computing apparatus. Various general-purpose computers may be used with programs written in accordance with the teachings herein, or, alternatively, it may be more convenient to construct a more specialized computer system to perform the required operations.

A number of embodiments have been described. Nevertheless, it will be understood that various modifications may be made. For example, the above description has primarily focused on hepatocytes and biological conditions that affect hepatocytes. It should however be noted that the same principles can be applied to other cells where cell-cell interactions are of interest or wherein the distribution of particular agents or sub-cellular components in the peripheral regions of the cells are of interest. The boundary regions have been identified above using a 3×3 matrix, but it should be realized that other sizes of matrices can be used as well, as can be determined based on the specific experimental conditions at hand. Accordingly, other embodiments are within the scope of the following claims.

1. A method for characterizing one or more cell features within one or more boundary regions of biological cells, the method comprising:
segmenting an image of one or more cells to identify cell boundaries for the individual cells in the image;
defining one or more boundary regions for the individual cells in the image, based on the identified cell boundaries; and
characterizing one or more cell features within the one or more defined boundary regions.

2. The method of claim 1, further comprising receiving said image of one or more cells in which a nucleus-marker and a cell shape-indicative marker identify the nucleus and an overall cell shape for cells in the image.

3. The method of claim 1, wherein the image comprises a digital representation of the one or more cells.

4. The method of claim 2, wherein the nucleus marker is a DNA marker and the cell shape-indicative marker is a non-specific protein marker or a cytoskeletal protein marker.

5. The method of claim 1, wherein the cells are hepatocyte cells.

6. The method of claim 1, wherein the image further contains a marker for each of the one or more cell features within the one or more boundary regions.

7. The method of claim 6, wherein the marker for the one or more cell features is selected from the group consisting of: an acting marker, an MRP2 marker, a BSEP marker, and a TGN marker.

8. The method of claim 1, wherein segmenting includes segmenting the image using a watershed algorithm.

9. The method of claim 1, wherein identifying boundary regions includes defining one or more of: a periphery region, a contact periphery region, a free periphery region, and a cell contact region, for the individual cells in the image.

10. The method of claim 9, wherein the periphery of a cell is identified in the image as a subset of pixels inside the cell for which a mask with a predetermined size centered on each of the pixel covers at least one of the cell's boundary pixels.

11. The method of claim 9, wherein the contact periphery of a cell is identified in the image as a subset of pixels inside the cell for which a mask with a predetermined size centered on each of the pixels covers at least one of the cell's boundary pixels and at least one boundary pixel of an adjacent cell.

12. The method of claim 9, wherein the free periphery of a cell is identified in the image as a subset of pixels that are periphery pixels but not contact periphery pixels.

13. The method of claim 9, wherein the cell contact is identified in the image as a pixels in a region between a pair of cells for which a mask with a predetermined size centered on each of the pixels covers at least one boundary pixel from each cell in the pair of cells.

14. The method of claim 1, wherein characterizing one or more cell features includes characterizing an acting level in the boundary regions.

15. The method of claim 14, further determining whether the cells possess and increased concentration of acting in the cell boundary regions.

16. A computer program product, stored on a machine-readable medium, for characterizing one or more cell features within one or more boundary regions of biological cells, comprising instructions operable to cause a computer to:
segment an image of one or more cells to identify cell boundaries for the individual cells in the image;
define one or more boundary regions for the individual cells in the image, based on the identified cell boundaries; and
characterize one or more cell features within the one or more defined boundary regions.

17. The computer program product of claim 16, further comprising instructions to receive said image of one or more cells in which a nucleus-marker and a cell shape-indicative marker identify the nucleus and an overall cell shape for cells in the image.

18. The computer program product of claim 16, wherein the image comprises a digital representation of the one or more cells.

19. The computer program product of claim 17, wherein the nucleus marker is a DNA marker and the cell shape-indicative marker is a non-specific protein marker or a cytoskeletal protein marker.

20. The computer program product of claim 16, wherein the cells are hepatocyte cells.

21. The computer program product of claim 16, wherein the image further contains a marker for each of the one or more cell features within the one or more boundary regions.

22. The computer program product of claim 16, wherein the marker for the one or more cell features is selected from the group consisting of: an acting marker, an MRP2 marker, a BSEP marker, and a TGN marker.

23. The computer program product of claim 16, wherein the instructions to segment include instructions to segment the image using a watershed algorithm.

24. The computer program product of claim 16, wherein the instructions to identify boundary regions include instructions to define one or more of: a periphery region, a contact periphery region, a free periphery region, and a cell contact region, for the individual cells in the image.

25. The computer program product of claim 24, wherein the periphery of a cell is identified in the image as a subset of pixels inside the cell for which a mask with a predetermined size centered on each of the pixel covers at least one of the cell's boundary pixels.

26. The computer program product of claim 24, wherein the contact periphery of a cell is identified in the image as a subset of pixels inside the cell for which a mask with a predetermined size centered on each of the pixels covers at least one of the cell's boundary pixels and at least one boundary pixel of an adjacent cell.

27. The computer program product of claim 24, wherein the free periphery of a cell is identified in the image as a subset of pixels that are periphery pixels but not contact periphery pixels.

28. The computer program product of claim 24, wherein the cell contact is identified in the image as a pixels in a region between a pair of cells for which a mask with a predetermined size centered on each of the pixels covers at least one boundary pixel from each cell in the pair of cells.

29. The computer program product of claim 16, wherein the instructions to characterize one or more cell features include instructions to characterize an acting level in the boundary regions.

30. The computer program product of claim 29, further comprising instructions to determine whether the cells possess and increased concentration of acting in the cell boundary regions.
